(19) United States
(12) Patent Application Publication
(10) Pub. No.: US 2001/0016577 A1
(43) Pub. Date: Aug. 23, 2001

(54) ORAL MUCOADHESIVE COMPOSITIONS CONTAINING GASTROINTESTINAL ACTIVES

(76) Inventors: DOUGLAS JOSEPH DOBROZSI, LOVELAND, OH (US); TODD EHREN VIENNEAU, ONTARIO (CA); CHRISTOPHER ROBERT MAYER, CINCINNATI, OH (US); KISHORKUMAR JIVANLAL DESAL, WEST CHESTER, OH (US)

Correspondence Address: THE PROCTER & GAMBLE COMPANY, PATENT DIVISION, HEALTH CARE RESEARCH CENTER, 8340 MASON-MONTGOMERY ROAD, MASON, OH, 45040, US

(21) Appl. No.: 09/361,933

(22) Filed: Jul. 27, 1999

Related U.S. Application Data
(60) Provisional application No. 60/097,576, filed on Aug. 24, 1998.

(51) Int. Cl. A61K 31/715

(52) U.S. Cl. 514/54; 514/57; 514/63; 514/503; 514/53; 514/867; 424/500; 424/485; 424/653; 424/488

(57) ABSTRACT

The present invention relates to mucoadhesive compositions comprising a safe and effective amount of a gastrointestinal active; from about 1.5% to about 10%, by weight of the composition, of a clay component; and from about 0.01% to about 1%, by weight of the composition, of a gum component. Alternatively, the clay component can be a titanium dioxide or a silicone dioxide component. The mucoadhesive compositions also preferably comprise up to about 2% by weight of the composition, of a non-ionic component such as methyl cellulose. The present invention also relates to methods of prevention and treatment of gastrointestinal tract disorders in humans or lower animals by orally administering a composition of the present invention.

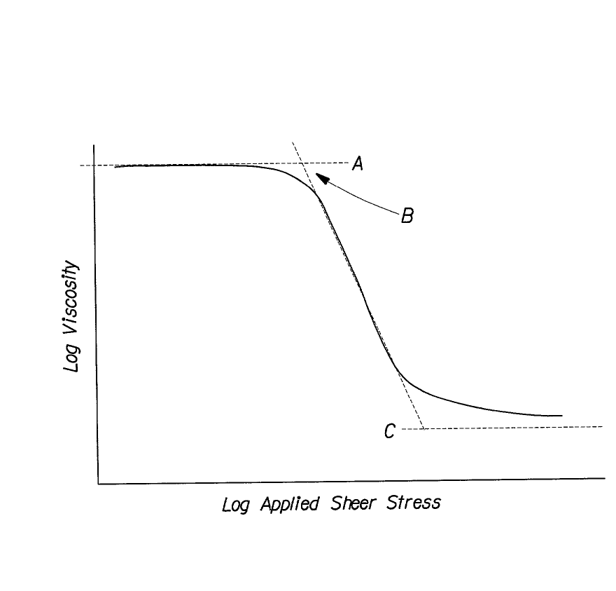

### CROSS-REFERENCE TO RELATED APPLICATIONS

This application claims the benefit of U.S. Provisional Application No. 60/097,576, filed Aug. 24, 1998.

### BACKGROUND OF THE INVENTION

The present invention relates to gastrointestinal pharmaceutical compositions suitable for oral administration to humans or lower animals which have improved retention. The compositions of the present invention are used widely to treat a variety of gastrointestinal disorders such as nausea, heartburn, and diarrhea. Some patent references, such as U.S. Pat. No. 4,940,695, to Coveney et al., which is incorporated herein by reference in its entirety, describe gastrointestinal pharmaceutical products containing bismuth such as Pepto-Bismol® (sold by the Procter & Gamble Company). Additionally, there are many other known gastrointestinal compositions. However, gastrointestinal compositions of the prior art have limited mucoadhesive properties. Prolonged coating is desirable as it protects the mucosa and underlying tissue from irritating or damaging agents and/or accelerates healing of inflamed or damaged tissue. Furthermore, prolonged coating also provides a matrix for enhanced delivery of the gastrointestinal active to the coated tissue resulting in higher efficacy and/or lower side effects.

Thus, there remains a need for a consumer acceptable gastrointestinal composition having enhanced mucoadhesion for exceptional coating and efficacy in the gastrointestinal tract. Through careful formulation, gastrointestinal compositions have been created which have both enhanced mucoadhesion as well as consumer acceptable viscosities and aesthetics. These mucoadhesive compositions comprise a gastrointestinal active, a clay/particulate component, a gum component, and optionally a non-ionic component. These and other formulations of the present invention will become readily apparent from the detailed description which follows. All percentages and ratios used herein are by weight and all measurements are at room temperature, unless otherwise indicated. As used herein, “ml” means milliliters, “mm” means millimeters, “mg” means mg, and “nm” means nanometers.

### SUMMARY OF THE INVENTION

The present invention relates to mucoadhesive compositions comprising a safe and effective amount of a gastrointestinal active; from about 1.5% to about 10%, by weight of the composition, of a clay component; and from about 0.01% to about 1%, by weight of the composition, of a gum component. Alternatively, the clay component can be a titanium dioxide or a silicone dioxide component. The mucoadhesive compositions also preferably comprise up to about 2% by weight of the composition, of a non-ionic component such as methyl cellulose. The present invention also relates to methods of prevention and treatment of gastrointestinal tract disorders in humans or lower animals by orally administering a composition of the present invention.

### BRIEF DESCRIPTION OF THE DRAWING

The FIG. (1/1) is an idealized rheogram which is useful for graphically showing a number of terms and concepts used in the present invention. The Figure is a plot of the Log of the applied shear stress to the Log of the viscosity. A represents the zero shear viscosity. B represents the yield stress, and C represents high shear viscosity.

### DETAILED DESCRIPTION OF THE INVENTION

The Figure plots the Log of the applied shear stress to the Log of the viscosity. The Figure is a representative rheogram resulting from the testing of a viscous shear thinning liquid material in a controlled-stress rheometer. In the stress ramp test, initially very low shear stress is applied to the sample, and gradually but continually the shear stress is increased, all the while determining the shear rate resulting in the sample. The Figure is useful for defining terms related to the viscosity and flow properties of liquid materials, particularly the shear thinning liquids claimed herein. The term “shear thinning”, as used herein, refers to a liquid having a higher viscosity when the applied shear is very low. At higher shear forces a shear thinning liquid has a lower viscosity. This characteristic low viscosity of a shear thinning composition under high shear stress is termed the “high shear viscosity” C. As the structure in the liquid is largely undisturbed by the initial low shear stress applied in the test, the composition's viscosity does not change to a large degree. However as shear stress is increased there will be a disproportionate increase in shear rate as the internal structure in the fluid breaks down, and correspondingly, viscosity decreases. The shear stress applied to the fluid at which rapid flow just begins to occur is termed the “yield stress”, or “yield value” B.

“Zero shear viscosity” is a measure of the internal structure in the liquid formulation and is the viscosity when stress below the yield stress is applied A. Zero shear viscosity may be accurately determined by the method of creep compliance using a sensitive, controlled stress rheometer. This method is described in the book “A Practical Approach to Rheology and Rheometry”, by Gebhard Schramm, 1994, page 107, which is incorporated herein by reference in its entirety. About 0.9 ml volume of the sample liquid is placed onto the plate of the rheometer (Haake RS150), and a 35 mm, 4 degree angle cone lowered to the measurement position. An equilibration shearing of about 20 per second is applied for about 10 seconds, then no stress is applied for 2 minutes. At the end of the 2 minutes, an instantaneous shear stress is applied and held constant for 5 minutes (hereinafter called the “creep stress”). This creep stress must be below the yield stress. A graph of the shear strain induced in the sample on the y axis against the time that the creep stress is applied on the x axis is generated. This graph will display an instantaneous large increase in shear strain at the beginning of the test, and after some period of curvature the graph will show that the shear strain increases proportionally in a straight line with time. The calculated slope of this shear strain-time line is divided into the applied creep stress to give a viscosity. As long as the creep stress is below the yield value of the liquid, then the viscosity determined in this manner is the zero shear viscosity. Other terms useful herein are defined below. Additionally, terms used in the art, as well as general concepts, are further described in “The Language of Colloid and Interface Science” by Laurier L. Schramm, American Chemical Society, 1993, which is incorporated herein by reference in its entirety.

The term “shear” as used herein is the rate of deformation of a fluid when subjected to a mechanical shearing stress. In simple fluid shear, successive layers of fluid move relative to each other such that the displacement of any one layer is proportional to its distance from a reference layer. The relative displacement of any two layers divided by their distance of separation from each other is termed the “shear” or the “shear strain”. The rate of change with time of the shear is termed the “shear rate”.

A certain applied force is needed to produce deformation in a fluid. For a plane area around some point in the fluid and in the limit of decreasing area the component of deforming forces per unit area that acts parallel to the plane is the “shear stress”.

The “viscosity” of a viscous material, also called viscosity index, is defined as the ratio of the shear stress applied into the material, divided by the rate of shear which results. Materials of a higher viscosity have a higher resistance to flow, or to forces which can induce flow, than a lower viscosity material. All viscosities listed herein are at a shear rate of about 50 per second unless otherwise indicated. Viscosities given herein can be measured in a controlled stress rotational viscometer, for Example Haake RS 150 namely by Haake GmbH, Karlsruhe, Germany, Carrimed CSL 500 Controlled Stress Rheometer by TA Instruments, New Castle, Del., and Rheometric SR5, by Rheometric Scientific, Piscataway, N.J.

The present invention relates to mucoadhesive oral formulations comprising colloidal suspensions which form a coating matrix on the epithelium of the gastrointestinal tract. The term “colloidal” as used herein refers to finely divided solid material in which the particles of TiO₂, SiO₂, and/or clay, are dispersed in a liquid phase and have a particle size of generally less than about 10 microns, or the particles have at least one dimension between about 1 and 1000 nm. The particle size of the solid particles of the present invention are of colloidal dimension, about 1 nm to 10 about microns, preferably 1,000 nm or smaller. The use of small particle sizes increases surface area for improved adsorption or bridging of the particle to mucin.

The term “colloidal suspension” as used herein refers to a system in which essentially solid colloidal particles are dispersed in a continuous phase of different composition or state, for example water. The colloidal suspensions of the present invention form a coating matrix on the mucosal epithelium of the gastrointestinal tract. Colloidal suspensions of the present invention should have high zero shear viscosity. Zero shear viscosity of the compositions herein should be at least about 2000 pascal seconds, preferably greater than about 7500 pascal seconds, more preferably greater than about 25,000 pascal seconds.

The term “gastrointestinal tract” as used herein refers to the mouth, pharynx, esophagus, upper and lower gastrointestinal tract, including the stomach and large and small intestines. The term “gastrointestinal disorder”, as used herein, encompasses any infection, disease or other disorder of the gastrointestinal tract which is treatable or preventable by oral administration of gastrointestinal actives. Examples of such disorders include heartburn, indigestion, nausea, vomiting, upset stomach, diarrhea, travelers' diarrhea, abdominal pain/cramping, constipation, gastritis, ulcers, and/or gastroesophageal reflux diseases. The term “gastrointestinal fluid” as used herein refers to saliva, gastric juice, intestinal fluid, and mixtures thereof.

The term “pharmaceutically-acceptable” as used herein means that the components present in the compositions of the present invention are compatible and suitable for oral administration to a human or lower animal. The term “compatible”, as used herein, means that the components of the pharmaceutical compositions are capable of being commingled with each other in a manner that there is no interaction which would substantially reduce the safety or pharmaceutical efficacy of the pharmaceutical compositions under ordinary use situations.

The term “mucoadhesive” or “bioadhesive” as used herein refers to the phenomenon where a natural or synthetic substance applied to a mucosal epithelium, adheres, usually creating a new interface, to the mucos layer. (CRC Critical Reviews in Ther Drug Carrier, Vol. 5 issue 1 (1988) pp. 21.) Generally, mucoadhesion can be achieved via physical or chemical processes or both. This mechanism is described in Journal of Controlled Release, Vol. 2 (1982) pp.257 and Journal of Controlled Release, Vol. 18 (1992) pp. 249. The above references are incorporated by reference herein in their entirety. The “mucoadhesive” compositions of the present invention have mucoretentive properties.

The term “mucoretentive” (or retentive) as used herein refers to a degree of resistance to the normal physiological propulsive mechanism involving both longitudinal and circular muscle fiber contraction, which transports substances through the gastrointestinal tract, i.e. resistance to peristalsis. Also, mucoretentive refers to a composition's degree of resistance to washing and dissolving forces of fluids in the gastrointestinal tract. The inventors employed a test which measures the tendency of a liquid formulation to coat onto gastrointestinal tissue and to resist the shear and rinsing forces of gastrointestinal fluid. This test was based on a method to evaluate the ability of gastrointestinal therapeutic formulations to bind and be retained on esophageal mucosa (L. R. Fitzpatrick et al., Gastroenterology, “A Comparison of Sucralfate and Bismuth Subsalicylate Formulation in Rabbit Esophageal Models”, Vol. 108, 1995 p. A94, incorporated herein by reference in its entirety). In this method, a freshly collected esophagus of a rabbit or a rat is cut into sections of about 2 cm in length. The tissue is everted onto a glass rod so that the mucosal surface is facing out. This mucosal surface can then be dipped into the formulation. Formulations with preferred rheology properties will tend to spread onto the mucosa and then form a coherent coating layer. The resistance to mechanical force and washing can be determined by vertically immersing the coated tissue into gastrointestinal fluid again and again by reciprocation. The amount of formulation which remains coated onto the tissue at the end of 30 rinses in gastrointestinal fluid or saliva has been determined to be a useful number for determining whether a formulation has mucoretentive properties. This may be quantified by measuring using a specific chemical analytical technique for a component of the formulation, or by incorporation of an easily measured, non-diffusing colloidal marker material into the formulation prior to testing. Mucoretentive compositions of the present invention have, after 30 rinses in simulated saliva, 80% of the initial amount still adhered to the tissues, preferably 85%, more preferably 90% or greater. Simulated saliva used for this retention test was adapted from Fusayama et al (Fusayama, T., Katayori, S., Nomoto, S., “Corrosion of gold and amalgam placed in contact with each other”, J. Dent. Res.42, 1963, 1183-1197) and contains on a mg/ml basis: KCl 0.4; NaCl 0.4; Na₂SO₄ 0.013; MgCl₂ 0.018; K₂HPO₄ 4.2; KH₂PO₄ 3.2; KOH 0.19; and bovine submaxillary mucin 4.0. The simulated saliva used for determination of triggered viscosity ratio was more concentrated to account for the dilution which occurs in the rheology test. It contained on a mg/ml basis 8.3 fold higher concentration of ingredients as the simulated saliva above, except that salivary mucin was at 83 mg/g. More preferably, when treating gastrointestinal disorders, compositions of the present invention have, after 30 rinses in simulated gastric fluid, 80% of the initial amount still adhered to the tissues, preferably 85%, more preferably 90% or greater. Simulated gastric fluid useful in this test can be made according to the instructions found in U.S. Pharmacopeia 23, 1995, US Pharmacopeial Convention, Rockville, Md., p. 2053, which is incorporated herein by reference in its entirety.

The term “glycoprotein” as used herein refers to a class of conjugated proteins comprising a protein with a carbohydrate group. Glycoproteins yield, in decomposition, a product frequently capable of reducing alkaline solutions of cupric oxide. Glycoproteins include mucins, mucoids, and the chondroproteins. The term “mucin” as used herein includes that which is contained in the saliva, gastrointestinal fluid, and/or associated with the surface of the gastrointestinal tissue. Mucin is produced within the body and provides lubrication and protection to the mucosal surfaces. It consists of a protein backbone, onto which are attached many polysaccharide chains. In the dry state, the mucin material is 70 to 80 percent by weight, carbohydrate. Mucin, with its high molecular weight, forms threadlike chains as much as 4-6 microns long, and may be effective in bridging of a colloidal suspension of particles which adsorbs it.

In order to provide suspensions with acceptable aesthetics, it is desirable for the suspensions to thin when they are shaken and/or poured into a spoon, cup, or other dosing apparatus. Such shaking and pouring subjects the suspensions to a shear rate of from about 10 to about 1,000 per second. Furthermore, when swallowed, a liquid is subjected to a shear rate of from about 10 to about 100 per second. It is also critical that the suspensions significantly thin when swallowed in order to achieve adequate spreading and coating of the gastrointestinal tract.

Specifically, when subject to constant shearing rate of about 100 per second, the present liquid compositions have a viscosity of less than about 1.5 pascal seconds, preferably less than about 0.75 pascal seconds, more preferably less than about 0.5 pascal seconds.

The solid particles of the present invention must be selected and formulated so that the contacting and mixing of a formulation of the present invention (hereinafter “the formulation”) to a mucosal surface such as the gastrointestinal tract triggers the conversion of the formulation to a more viscous gel-like mixture. In other words, after the formulation mixes with gastrointestinal fluid, the viscosity of the formulation is greater than the viscosity of either the formulation prior to mixing, or the gastrointestinal fluid mixture alone.

The value of a formulation's triggered viscosity ratio (“T”) is useful in determining the degree to which a composition exhibits the above described gelling characteristic. The formula and procedure for determining the triggered viscosity ratio is set forth below.

It is desirable for the compositions of the present invention to exhibit a triggered viscosity ratio of at least about 1.2, more preferably at least 1.4, and most preferably at least about 1.5 where the triggered viscosity is defined by the following formula:

$$T = \frac{{η}_{g}}{{η}_{f}}$$

where η_{g}=viscosity of the gel and

where η_{f}=viscosity of the formulation of the present invention

As used herein, the term “gel” describes the substance resulting from the combination of a mucin saliva mixture and the formulation. For determining the triggered viscosity ratio herein, the mucin saliva mixture is formed by mixing a commercial supply of mucin and a concentrated form of saliva. For use in the present test, mucin saliva comprises, on a mg/ml basis, KCl 3.32, NaCl 3.32, Na₂SO₄ 0.108, MgCl₂ 0.150, K₂HPO₄ 34.86, KH₂PO₄ 26.56, KOH 1.57, and bovine submaxillary mucin 83. The bovine submaxillary mucin is commercially available from Sigma Chemical Co., St. Louis, Mo., as bovine submaxillary mucin type I, catalog #M4503.

The triggered viscosity ratio of a formulation can be determined by the following method. First, the viscosity of the formulation (η_{f}) is determined in a rheometer using a shear rate of 50 per second. For the determination of η_{f}, 0.9 ml of the formulation is placed onto the plate of a Haake RS150 rheometer. The temperature is controlled in the range of typical room temperature, about 23° C. A cover is used on the measuring system to prevent evaporation of water from the sample during the test. A 35 mm diameter, 4 degree angle cone measuring system is lowered onto the sample, and an equilibration shearing of approximately 20 per second is applied for 20 seconds. After a rest period wherein no stress is applied for 2 minutes, a constant shearing rate of 50 per second is applied for 65 seconds. The viscosity η_{f} is read from the instrument at the 60 second time point.

For the determination of η_{g}, 0.5 ml of the mucin saliva mixture defined above is combined with 4.5 ml of the formulation and the two are gently mixed together for 5 minutes. The mixture is then loaded onto the plate of the same rheometer used for the measurement of if, except that the temperature is controlled at the normal body temperature of a human, 37° C. An identical rheometer measurement program is used as for determination of η_{f}. The triggered viscosity factor is calculated from η_{f} and η_{g} as described above.

SEDIMENTATION VOLUME RATIO: Another essential feature of the compositions of the present invention is that the compositions have a sedimentation volume ratio of greater than about 0.90, preferably greater than about 0.95, more preferably greater than about 0.98, and even more preferably about 1 (after about 48 hours). Sedimentation volume ratio is determined by carefully filling a sample of the formulation into a clear glass graduated cylinder, capping the cylinder to prevent any evaporation, and allowing the formulation to remain undisturbed and free from significant vibration. After at least 48 hours, total occupied volume in the cylinder (Vₒ) and the ultimate volume of any sediment which may have formed by settling of components of the suspension below the total volume (Vᵤ) is determined. (This procedure is explained in “Coarse Dispersions”, Chapter 18 in “Physical Pharmacy”, A. Martin, Lea and Febiger, Malvern, Pa., 1993, page 480). The sedimentation volume ratio is then the ratio of the ultimate volume to the occupied volume (Vᵤ/Vₒ).

COMPONENTS: The pharmaceutical compositions of the present invention are formulated to provide a safe and effective amount of the components defined below. Specifically, as used herein the phrase “safe and effective amount” means an amount of particulate component, clay, gum component, and/or gastrointestinal active, high enough to significantly (positively) modify the condition to be treated or to effect the desired result, but low enough to avoid serious side effects (at a reasonable benefit/risk ratio), within the scope of sound medical judgment. The safe and effective amount, will vary with the particular condition or disease being treated, the age and physical condition of the patient being treated, the severity of the condition, the duration of treatment, the nature of concurrent therapy, the specific form of the particles or active agent employed, the particular vehicle from which the particles or active agent is applied, and like factors within the knowledge of the attending physician or individual having ordinary skill in the art.

Clays: The clay is present at a level of from about 1.5% to about 10% by weight of the composition, preferably from about 2.5% to about 6.5%, more preferably from about 3.5% to about 4.5% by weight. Clays are composed of fine particles of clay minerals which are layer-type hydrous (containing structural hydroxyl groups) silicates of aluminum, magnesium, potassium, iron, and other less abundant elements, particularly alkalis and alkaline earth metals. Preferred are silicates of aluminum, magnesium and iron. More preferred are silicates of aluminum. Preferred is magnesium aluminum silicate (or aluminum magnesium silicate), occurring naturally in such smectite minerals as colerainite, saponite, and sapphirine. Refined magnesium aluminum silicates useful herein are readily available as Veegum, manufactured by R. T. Vanderbilt Company, Inc.

Clay may also contain varying amounts of non-clay minerals such as quartz, calcite, feldspar, and pyrite. Preferred clays useful herein are water swellable clays.

The term “clay”, as used herein, includes but is not limited to kaolin minerals such as kaolinite, china clay, dickite, nacrite, halloysite; serpentine minerals such as lizardite, halloysite, chrysotile, antigorite, carlosturanite, amestite, cronstedite, chamosite, berthierine, garierite; talc; pyrophyllite; ferripyrophyllite; smectites such as montmorillonites, beidellite, nontronite, hectorite, saponite, sauconite, medmontite, pimelite, bentonite; illite minerals such as ledikete, bravaisite, degraded mica, hydromica, hydromuscovite, hydrous illite, hydrous mica, K-mica, micaceous clay, and sericite; mica such as pegmatite, muscovite, and phlogopite; brittle mica such as margarite, and clintonite; glauconite; celadonite; chlorite and vermiculite such as pennine, clinochlore, chamosite, nimite, baileychlore, donbassite, cookite, sudoite, franklinfurnaceite; palygorskite and sepiolite minerals such as attapulgite; allophane and imogolite; mixed layer clay minerals such as talc-chlorite; and mixtures thereof.

Preferred clays are selected from the group consisting of kaolin minerals, smectites, mica, and mixtures thereof. More preferred are clays selected from the group consisting of laponite, bentonite, hectorite, saponite, montmorillonites, and mixtures thereof.

Any of the available forms are acceptable for use in the present invention such as colloidal clays, for example magnesium aluminosilicate, magnesium bentonite, attapulgite, sodium bentonite magma, etc.

Clays which are useful in the present invention include both mined, naturally occurring clays as well as synthetic clays. The clays must be pharmaceutically-acceptable. A more detailed description of the clays and clay minerals useful herein can be found in the following three references, each of which is incorporated by reference in its entirety: Kirk-Othmer, Encyclopedia of Chemical Technology, Fourth Edition, Vol. 6, pages 381-423; Dell, D. J., “Smectite Clays in Personal Care Products”, Cosmetics & Toiletries, Vol. 108, May 1993, pages 79-85; and Theng B. K. G., Formation and Properties of Clay-Polymer Complexes, Developments in Soil Science, Vol. 9.

Particulate Component: Throughout the present invention, a particulate component selected from the group consisting of silicone dioxide components and/or titanium dioxide components can be substituted for the clays. Mixtures of clays and particulate components can also be used. However, clays are preferred.

Silicon Dioxide (Silica): The silicon dioxide is present at a level of from about 2% to about 50% by weight of the composition, preferably from about 3% to about 20%, more preferably from about 4% to about 9% by weight. Any of the available forms are acceptable for use in the present invention such as fumed silicon dioxide, precipitated silicon dioxide, colloidal silicon dioxide, coacervated or gels. Fumed silicon dioxide is especially effective at from about 5% to about 20% by weight. These silica particles may be chemically surface modified, for example with methyl siloxane, to enhance the tissue barrier properties of the coating to hydrophilic substances.

Titanium Dioxide: The titanium dioxide is present at a level of from about 2% to about 50% by weight of the composition, preferably from about 3% to about 20%, more preferably from about 4% to about 9% by weight. Any of the available pharmaceutical grade forms of titanium dioxide are acceptable for use in the present invention as long as such form achieves the mucin interaction described by the present invention and efficiently achieves acceptable sedimentation volume ratio specified herein. Such forms include rutile, anatase crystalline form, amorphous form, and any other form which is acceptable for the purposes of the present invention. These titanium dioxide particles may be preferably chemically surface modified, for example with alumina, silica, or other stabilizing agent, to enhance the tissue barrier properties of the coating to hydrophilic substances.

The two major processes used in manufacturing titanium dioxide are sulfate and chloride. The processes are usually followed by modification of particle surfaces with treatments and coatings. Certain additives are used for modifying the titanium dioxide which affect the surface properties, for example zinc salts that form zinc titanate at the crystal surfaces, alumina, silica, and titania coatings in aqueous dispersions. In addition titanium dioxide can be further modified by organic surface treatments. Organic surface treatments include surface active agents, saturated and unsaturated fatty acid, oleic acid, dehydrated castor oil acid, and derivatives of these compounds, and mixtures thereof. Further details of surface properties of titanium dioxide are found in H. S. Ritter, “Surface Properties of Titanium Dioxide Pigments”, Pigment Handbook, Chemical Division, PPG Industries (1973), Volume 3, 169-84.

Small particle size titanium dioxide is preferred, i.e. titanium dioxide having a mean particle size of less than about 1 micron. Preferably, uncoated titanium dioxide having mean particle sizes of from about 20 nm to about 400 nm, even more preferably about 50 nm are used. Uniform spherical and uncoated titatium dioxide useful herein, having primary particle size diameters of approximately 50 nm, can be synthesized via the method disclosed in N. Kallay and E. Matijevic, Langmvir 1, p. 195, 1985, which is incorporated herein by reference in its entirety.

Gum Component: The term “gum component”, as used herein, refers to a component selected from the group consisting of xanthan gum, guar gum, locust beam gum, carrageenans, tragacanth, and carbomer. Preferred is xanthan gum. Xanthan gum is available as “Rhodigel” from R. T. Vanderbilt Corp., Norwalk, Conn. The gum component should be included in the present invention from about 0.01% to about 1% by weight of the composition, preferably from about 0.05% to about 0.5%, more preferably from about 0.1% to about 0.3%. The compositions of the present invention should comprise clay and gum component such that the clay to gum component ratio is from about 10:1 to about 100:1. Preferred, the clay to gum component ratio is from about 12:1 to about 65:1, more preferred from about 35:1 to about 45:1.

The Gastrointestinal Active: The compositions of the present invention also comprise a safe and effective amount of at least one oral pharmacologically active gastrointestinal agent. For example, the compositions of the present invention may comprise from about 0.01% to about 50% of gastrointestinal active, by weight of the composition, preferably from about 0.1% to about 20%, more preferably from about 0.5% to about 5%, and most preferably from about 0.7% to about 3%, by weight of the composition. Examples of gastrointestinal agents useful in the present invention include but are not limited to: antacids, anticholinergics, bismuth compounds, prostaglandin analogs, H₂-receptor antagonists, laxatives, gastroprotectants, gastrokinetic and prokinetic agents, proton pump inhibitors, antidiarrheals, agents which are bacteriostatic or bactericidal to the ulcer-inducing organism Heliobacter pylori, topical anesthetics, topical analgesics, and polyanionic materials useful for the treatment of ulcers and other gastrointestinal disorders. Preferred for use herein are gastrointestinal agents including antacids, H₂-receptor antagonists, gastroprotectants, proton pump inhibitors, antidiarrheals, and polyanionic materials useful for the treatment of ulcers and other gastrointestinal disorders. More preferred are antacids, gastroprotectants, and antidiarrheals.

Examples of anticholinergics useful herein include but are not limited to atropine, clidinium and dicyclomine. Examples of antacids useful herein include but are not limited to aluminum hydroxide. Other examples of antacids can be found in 21 CFR 331.11 which is incorporated herein by reference. Examples of H₂-receptor antagonists useful herein include but are not limited to cimetidine, famotidine, nizatidine and ranitidine. H₂-receptor antagonists compounds useful herein are further disclosed in U.S. Pat. No. 5,294,433, Singer et al., issued Mar. 15, 1994, which is herein incorporated by reference in its entirety. Examples of laxatives useful herein include but are not limited to bisacodyl, picosulfate, and casanthrol. Other examples of laxatives can be found in the Federal Registry, vol. 50, no. 10, Jan. 15, 1985, pages 2152-2158 which is incorporated herein by reference. Examples of gastroprotectants useful herein include but are not limited to sucralfate and sucralfate humid gel. Examples of gastrokinetic and prokinetic agents useful herein include but are not limited to cisapride, metoclopramide and eisaprode. Examples of proton pump inhibitors useful herein include but are not limited to omeprazole. Examples of antidiarrheals useful herein include but are not limited to diphenoxylate bismuth subsalicylate, attapulgite, and loperamide. Examples of agents which are bacteriostatic or bactericidal to the ulcer-inducing organism Heliobacter pylori useful herein include but are not limited to amoxicillin, metronidazole, erythromycin, and nitrofurantoin. These and other agents for treating H. pylori are disclosed in U.S. Pat. No. 5,256,684, to Marshall, issued Oct. 26, 1993, which is incorporated herein by reference in its entirety. Examples of topical anesthetics useful herein include but are not limited to lidocaine and benzocaine. Examples of topical analgesics useful herein include but are not limited to menthol, acetaminophen, salicylates including aspirin (acetylsalicylic acid), , ibuprofen and naproxen. These are described more completely in the following: U.S. Pat. No. 4,749,720 to Sunshine et al. issued Jun. 7, 1988, U.S. Pat. No. 4,749,711 to Sunshine et al. issued Jun. 7, 1988, U.S. Pat. No. 4,749,697 to Sunshine et al. issued Jun. 7, 1988, U.S. Pat. No. 4,783,465 to Sunshine et al., issued Nov. 8, 1988, U.S. Pat. No. 4,619,934 to Sunshine et al., issued Oct. 28, 1986, U.S. Pat. No. 4,552,899 to Sunshine et al. issued Nov. 12, 1985, all of which are incorporated by reference herein, in their entirety. Examples of polyanionic materials useful for the treatment of ulcers and other gastrointestinal disorders useful herein include but are not limited to amylopectin, carragenan, sulfated dextrins, inositol hexaphosphate, or other similar agents.

Also extracts of plants or other natural substances known to be effective in any of the above disorders can be delivered from the mucoadhesive matrix. Examples of each of the aforementioned gastrointestinal actives are further described, along with appropriate dosages, in Facts and Comparisons, 1998, pp. 242-260, 291-326h, and 601-607 which is incorporated herein by reference in its entirety.

Bismuth: The preferred gastrointestinal agent for use in the present invention is bismuth. As used herein, the quantity of bismuth is by weight of elemental bismuth. In the present invention, the term “bismuth”, as used herein, includes bismuth in the form of a pharmaceutically-acceptable salt, bismuth or bismuth salt in the form of an organic or other complex which contains bismuth as an active ingredient, and mixtures thereof. Such organic complexes include 2,2′-spirobi[ 1,,32-benzodoxabismole]. Preferred is a pharmaceutically-acceptable salt. Such bismuth salts include bismuth aluminate, bismuth subcarbonate, bismuth subcitrate, bismuth citrate, bismuth ranitidine citrate complex, tripotassium dicitrato bismuthate, bismuth subgalate, bismuth subnitrate, bismuth tartrate, bismuth subsalicylate, and mixtures thereof. Bismuth citrate, bismuth ranitidine citrate complex, bismuth subcitrate, tripotassium dicitrato bismuthate, bismuth tartrate, bismuth subsalicylate, and mixtures thereof are preferred bismuth salts for use in this invention. When used in the compositions of the present invention, bismuth preferably comprises from about 0.8% to about 7% of bismuth, more preferably from about 1% to about 3%, by weight of the composition.

Non-ionic Component: Non-ionic components are optionally present at a level of from about 0.0% to about 2% by weight of the composition, preferably from about 0.05% to about 0.5%. Non-ionic components useful in the present invention are selected from the group consisting of methyl cellulose, ethyl cellulose, hydroxyethyl cellulose, hydroxypropyl cellulose, hydroxypropyl methyl cellulose, carboxymethyl cellulose, polyvinyl pyrrolidone, acacia, propylene glycol alginate, sodium alginate, and sodium starch glycolate. Preferred is methyl cellulose.

Optional Consistency Aids: Optionally, consistency aids are present at a level of from about 0.1% to about 50% by weight of the composition, preferably from about 1% to about 30%, more preferably from about 2% to about 20% by weight. These consistency aids are low molecular weight mono and polyols and are selected from the group consisting of monosaccharides such as glucose (dextrose), fructose (levulose); disaccharides such as sucrose, lactose, maltose, cellobiose and other sugars, ribose, glycerine, sorbitol, xylitol, inositol, propylene glycol, galactose, mannose, xylose, rhamnose, glutaraldehyde, invert sugars, ethanol, honey, mannitol, polyethylene glycol, glycerol, and mixtures thereof; preferably the polyols are selected from the group consisting of honey, sorbitol, glycerine, glycerol and mixtures thereof.

These compounds help provide physical stability to the compositions. In addition these compound, such as low molecular weight polyols, are preferred for providing the proper consistency of the composition prior to administration so that an optimal degree of spreading over the mucosa is achieved after administration. Specifically, these polyols will reduce or delay the rate at which the particulates in the dispersions bridge or are adsorbed by the glycoproteins of the mucosa. This permits the composition to better spread and coat the tissue before triggering causes the viscosity of the composition to increase.

Pharmaceutically-Acceptable Excipients: The liquid phase of the colloidal suspensions of the present invention is generally water. These compositions comprise from about 5% to about 98%, preferably from about 70% to about 95%, by weight of the composition of water.

Optionally, these aqueous compositions also contain suitable amounts of preservatives, emulsifying agents, suspending agents, diluents, natural or artificial sweeteners, taste-masking agents, coloring agents, and flavoring agents, to provide a palatable and pleasant looking final product. Also, compositions may also comprise antioxidants, for example, butylated hydroxy anisole or butylated hydroxy toluene, and preservatives, for example, methyl or propyl paraben or sodium benzoate, to prolong and enhance shelf life. Antimicrobials may also optionally be added to the compositions of the present invention.

Additionally, optionally useful herein are sensory agents selected from the group consisting of coolants, salivating agents, warming agents. If present, these agents comprise from about 0.001% to about 10%, preferably from about 0.1% to about 1%, by weight of the composition. Suitable cooling agents include but are not limited to paramenthan carboxyamide agents such as N-ethyl-p-menthan-3-carboxamide, menthol, 3-1-menthoxypropane-1,2-diol, menthone glycerol acetal, menthyl lactate, and cyclic sulphones and sulphoxides, Salivating agents useful herein include Jambu® manufactured by Takasago Perfumery Co., Ltd., Tokyo, Japan. Warming agents include capsicum and nicotinate esters, such as benzyl nicotinate.

The compositions of the present invention also preferably but optionally contain from about 0.005% to about 3%, preferably from about 0.07% to about 1.5% of substituted or unsubstituted short chain organic acids or water soluble salts thereof, including citric, tartaric, acetic, malic, maleic, and succinic to provide consistent dispersion of the solid particles thereby improving stability. Specific examples of pharmaceutically acceptable carriers and excipients that may be used to formulate oral dosage forms, are described in U.S. Pat. No. 3,903,297, Robert, issued Sep. 2, 1975, incorporated by reference herein.

### METHODS OF USE

The compositions of the present invention should be orally administered at a safe and effective amount for the treatment and/or prevention of gastrointestinal disorders. An example of a method of treatment and/or prevention may comprise the oral administration to a human or lower animal subject from about 1 ml to about 100 ml, preferably from about 20 ml to about 50 ml per dose of the compositions of the present invention. The compositions of the present invention are dosed as needed, preferably not more than 30 times per day, more preferably not more than 15 times per day, and most preferably not more than 8 times per day. Such oral administration of the compositions of the present invention provides a superior coating system (the clay and/or particulate component, and gum component combination claimed herein) which may help protect the mucosa and underlying tissue from irritating or damaging agents and/or accelerate healing of inflamed or damaged tissue. Furthermore, this superior coating system allows for enhanced delivery of the gastrointestinal active to the coated tissue resulting in higher efficacy and/or lower side effects.

### METHOD OF MAKING

These compositions can be prepared by the following method: Using high shear mixing, disperse the clays and/or the particulate components into water. With continued mixing, add the gum component. Where included in the formulation, add one or more non-ionic components with mixing (it may be useful to first prepare a diluted solution of the non-ionic components). Where included in the formulation, sweeteners such as corn syrup, or sorbitol are now added. These steps form the main mixture. Separately, combine water and color to form a color premix. Prepare a second premix by combining the gastrointestinal active(s) along with other optional ingredients such as flavors, excipients, preservatives, etc. with a small quantity of water. Create a colored main mixture by adding the color premix to the main mixture with mixing. Add the second premix slowly to the colored main mixture with gentle blending. Add water to bring to final batch weight. Mix until homogeneous.

The following non-limiting examples illustrate the methods and uses of the present invention.

### EXAMPLES

| | Examples (w/w%) | | | | |
|---|---|---|---|---|---|
| Ingredient | No.1 | No.2 | No.3 | No.4 | No.5 |
| Gastrointestinal Active | 10% | 5% | 2% | 0.1% | 0.5% |
| Non-ionic Component | 0.3% | — | — | 0.1% | 0.2% |
| Gum Component | 0.1% | 0.1% | 0.06 | 0.13% | 0.1% |
| Clay | 4.3% | — | — | 2.5% | 4.0% |
| Particulate Component | — | 8% | 5% | 2% | — |
| Flavor | 0.09% | 0.1% | 0.09% | 0.1% | 0.09% |
| Bulk sweetener | — | 30% | — | — | — |
| Bulk Artificial | — | — | — | — | 25% |
| sweetener | | | | | |
| Artificial sweetener | 0.06% | — | 0.06% | 0.06% | — |
| Preservative | 0.038% | 0.040% | 0.035% | 0.037% | 0.041% |
| Color | 0.012% | 0.012% | 0.012% | 0.012% | 0.012% |
| Purified water | Q.S. to | Q.S. to | Q.S. to | Q.S. to | Q.S. to |
| | 100% | 100% | 100% | 100% | 100% |
| | Examples (w/w%) | | | | |
| Ingredient | No.6 | No.7 | No.8 | No.9 | No.10 |
| Bismuth Subsalicylate | 1.8% | — | — | — | — |
| Ranitidine | — | 0.033% | — | — | — |
| Sucralfate | | | | | |
| Phenol | — | — | — | — | — |
| Calcium Carbonate | — | 1.7% | — | — | 3.3% |
| Methylcellulose | 0.2% | — | — | 0.1% | 0.2% |
| Xanthan Gum | 0.1% | 0.1% | 0.06 | 0.13% | 0.13% |
| Magnesium | 4.3% | 2% | 2.5% | — | — |
| Aluminum Silicate | | | | | |
| Titanium Dioxide | — | — | — | — | 8% |
| Silicon Dioxide | — | 2% | — | — | — |
| Laponite | — | — | — | 3.5% | — |
| Methyl Salicylate | 0.09% | 0.1% | 0.09% | 0.09% | 0.09% |
| Salicylic Acid | 0.07% | 0.07% | 0.07% | 0.07% | 0.07% |
| Sodium salicylate | 0.06% | 0.06% | 0.06% | 0.06% | 0.06% |
| High fructose corn | — | 30% | — | — | — |
| syrup | | | | | |
| Sorbitol | — | — | — | — | 25% |
| Sodium Saccarin | 0.06% | — | 0.06% | 0.06% | — |
| Benzoic Acid | 0.025% | 0.025% | 0.025% | 0.025% | 0.025% |
| Sorbic Acid | 0.013% | 0.013% | 0.013% | 0.013% | 0.013% |
| Color | 0.012% | 0.012% | 0.012% | 0.012% | 0.012% |
| Purified water | Q.S. to | Q.S. to | Q.S. to | Q.S. to | Q.S. to |
| | 100% | 100% | 100% | 100% | 100% |

### Example 11

A man having diarrhea coupled with nausea and vomiting orally takes a 30 ml dose of a composition as shown in Example 6. Shortly, the symptoms have subsided and the man feels normal again.

### Example 12

A woman overindulges at dinner and therefore has indigestion. She orally takes a 40 ml dose of a composition as shown in any of Examples 1-8 and 10, and her indigestion is relieved.

### Example 13

A woman knows that eating spicy foods routinely causes her heartburn. Therefore, each time prior to indulging in such foods, she orally takes a 35 ml dose of a composition as shown in any of the Examples 1-8 and 10. Such action coats her gastrointestinal tract and prevents her from suffering from heartburn as a result of eating the spicy food.

What is claimed is:

1. A per oral, oral, mucoretentive, aqueous liquid pharmaceutical composition comprising:
a. a safe and effective amount of a gastrointestinal active;
b. from about 1.5% to about 10%, by weight of the composition, of clay; and
c. from about 0.01% to about 1% by weight of the composition, of a gum component selected from the group consisting of xanthan gum, guar gum, locust beam gum, carrageenans, tragacanth, and carbomer;
and wherein the clay and the gum component are at a ratio of from about 10:1 to about 100:1.

2. The pharmaceutical composition according to claim 1 further comprising a non-ionic component selected from the group consisting of methyl cellulose, ethyl cellulose, hydroxyethyl cellulose, hydroxypropyl cellulose, hydroxypropyl methyl cellulose, carboxymethyl cellulose, polyvinyl pyrrolidone, acacia, propylene glycol alginate, sodium alginate, and sodium starch glycolate.

3. The pharmaceutical composition according to claim 1 wherein the clay and the gum component are at a ratio of from about 35:1 to about 65:1.

4. The pharmaceutical composition according to claim 1 wherein the pharmaceutical composition has a zero shear viscosity of at least about 2000 pascal seconds.

5. The pharmaceutical composition according to claim 1 wherein the pharmaceutical composition has a sedimentation volume ratio of greater than about 0.90 when measured after about 48 hours.

6. The pharmaceutical composition according to claim 1 wherein the pharmaceutical composition has a triggered viscosity ratio of at least about 1.2.

7. The pharmaceutical composition according to claim 2 comprising from about 3.5% to about 4.5%, by weight of the composition, of the clay and wherein the pharmaceutical composition has a sedimentation volume ratio of greater than about 0.95 when measured after about 48 hours.

8. The pharmaceutical composition according to claim 2 wherein the gastrointestinal active is one or more bismuth salts.

9. The pharmaceutical composition according to claim 3 comprising from about 0.05% to about 0.5%, by weight of the composition, of the gum component.

10. A per oral, oral, mucoretentive, aqueous liquid pharmaceutical composition comprising:
a. a safe and effective amount of a gastrointestinal active;
b. from about 2% to about 50%, by weight of the composition, of a particulate component selected from the group consisting of silicone dioxide and titanium dioxide; and
c. from about 0.01% to about 1% by weight of the composition, of a gum component selected from the group consisting of xanthan gum, guar gum, locust beam gum, carrageenans, tragacanth, and carbomer.

11. The pharmaceutical composition according to claim 10 further comprising a non-ionic component selected from the group consisting of methyl cellulose, ethyl cellulose, hydroxyethyl cellulose, hydroxypropyl cellulose, hydroxypropyl methyl cellulose, carboxymethyl cellulose, polyvinyl pyrrolidone, acacia, propylene glycol alginate, sodium alginate, and sodium starch glycolate.

12. The pharmaceutical composition according to claim 10 wherein the pharmaceutical composition has a zero shear viscosity of at least about 2000 pascal seconds.

13. The pharmaceutical composition according to claim 10 wherein the pharmaceutical composition has a viscosity of less than about 0.75 pascal seconds when subjected to a constant shearing rate of about 100 per second.

14. The pharmaceutical composition according to claim 10 wherein the pharmaceutical composition has a triggered viscosity ratio of at least about 1.2.

15. The pharmaceutical composition according to claim 10 wherein the pharmaceutical composition has a sedimentation volume ratio of greater than about 0.90 when measured after about 48 hours.

16. A pharmaceutical composition according to claim 11 comprising from about 5% to about 20%, by weight of the composition, of the particulate component.

17. A pharmaceutical composition according to claim 11 comprising from about 0.05% to about 0.5%, by weight of the composition, of the gum component.

18. A method for treatment or prevention of a gastrointestinal disorder in a human or lower animal subject comprising coating the gastrointestinal tract by orally or per orally administering to the subject an effective amount of a composition according to claim 1.

19. A method for treatment or prevention of a gastrointestinal disorder in a human or lower animal subject comprising coating the gastrointestinal tract by orally or per orally administering to the subject an effective amount of a composition according to claim 8.

20. A method for treatment or prevention of a gastrointestinal disorder in a human or lower animal subject comprising coating the gastrointestinal tract by orally or per orally administering to the subject an effective amount of a composition according to claim 10.
